(19) United States
(12) Patent Application Publication
(10) Pub. No.: US 2024/0197808 A1
(43) Pub. Date: Jun. 20, 2024

(54) COMPOSITIONS FOR MITIGATING THE EFFECTS OF ALCOHOL CONSUMPTION AND METHODS RELATED THERETO

(71) Applicant: LIFTED BEVERAGES CORPORATION, San Diego, CA (US)

(72) Inventor: Reza Tirgari, San Diego, CA (US)

(21) Appl. No.: 18/510,162

(22) Filed: Nov. 15, 2023

Related U.S. Application Data
(60) Provisional application No. 63/426,679, filed on Nov. 18, 2022.

(51) Int. Cl. A61K 36/82; A61K 31/197; A61K 31/198; A61K 31/375; A61K 31/4415; A61K 31/455; A61K 31/51; A61K 31/52; A61K 31/7004; A61K 31/714; A61K 33/30; A61K 36/258; A61K 36/9066

(52) U.S. Cl. CPC A61K 36/82; A61K 31/197; A61K 31/198; A61K 31/375; A61K 31/4415; A61K 31/455; A61K 31/51; A61K 31/52; A61K 31/7004; A61K 31/714; A61K 33/30; A61K 36/258; A61K 36/9066

(57) ABSTRACT

Compositions and methods for mitigating and preventing the effects of alcohol consumption are provided. A first composition can be ingested by an individual prior to or within six hours of alcohol consumption. A second composition can be ingested by the individual either at the onset of negative symptoms associated with alcohol consumption or the morning following alcohol consumption. The first and second compositions can be ingested individually, or in combination. When both the first and second composition are consumed, the first composition is to be consumed prior to the second composition, and both are to be consumed in a timely manner. Both compositions can be administered orally. The compositions can each be administered as a fluid solution. Only one serving of each composition is required to achieve the desired effects. Results are strengthened when both compositions are consumed in combination.

### CROSS-REFERENCE TO RELATED APPLICATIONS

The present application claims priority to U.S. Provisional Patent Application No. 63/426,679, filed Nov. 18, 2022, which is incorporated herein by reference in its entirety.

### FIELD

The present subject matter broadly relates to compositions and methods for mitigating the effects of alcohol consumption. The present subject matter further relates to administering and/or ingesting a combination of ingredients, including but not limited to, vitamins, minerals, amino acids, antioxidants, and extracts.

### BACKGROUND

A myriad of negative physiological and psychological effects can follow the consumption of alcohol. More pronounced negative effects are known as a “hangover” effect from alcohol consumption typically manifest several hours after consumption of alcohol. Hangover-related symptoms can last for several hours or even exceed more than 24 hours in some instances. Symptoms experienced following alcohol consumption include, but are not limited to, headache, lethargy, cognitive impairment, insomnia, dehydration, dry mouth, dizziness, fatigue, and nausea. Alcohol hangovers vary greatly in severity and can correlate strongly with the degree of alcohol intoxication preceding the hangover.

Alcohol is a carbohydrate and is metabolized though various body metabolism cycles in the liver to form carbon dioxide and water. Specifically, clearance of alcohol from a drinker's body is typically achieved primarily through metabolism of alcohol to acetaldehyde, which is subsequently metabolized to acetic acid, and then to carbon dioxide. Through this process, alcohol is eventually removed from the blood and tissues, but not before the lingering acetaldehyde in the organs and tissues causes the hangover symptoms described herein.

It is generally known that a number of precautions can be taken contemporaneously with alcohol consumption and after in order to mitigate the risk of suffering from undesirable effects associated with alcohol consumption. For example, due to the dehydrating effect of alcohol, it is recommended to drink copious amounts of water when consuming alcohol and following consumption. It is also well known that consuming food with alcohol can mitigate intoxication and reduce the potential for any number of negative effects, including a hangover. Commonly, individuals take medications, such as ibuprofen or acetaminophen in order to avoid the onset of hangover symptoms following alcohol consumption. Though these medications can be effective at treating hangover symptoms, some may cause liver damage or exacerbate liver damage caused by alcohol or acetaldehyde.

Thus, there is a need for compositions to alleviate the negative effects associated with alcohol consumption in a safe and effective manner. It would be further advantageous to provide compositions that can be easily administered (e.g., through oral ingestion), which target specific needs during all phases of alcohol metabolism. Specifically, it would be desirable to provide a first composition which targets the initial phases of alcohol metabolism so as to buffer the body from the negative effects associated with alcohol consumption. Additionally, it would be desirable to provide a second composition which targets the later phases of alcohol metabolism so as to mitigate and alleviate symptoms experienced after a user ceases drinking alcohol. It would also be further advantageous to provide hangover remedy compositions that taste good, are nutritious, and boost energy to compensate for energy loss associated with alcohol consumption.

### SUMMARY

The present subject matter provides compositions and methods for mitigating, preventing, and/or alleviating the effects of alcohol consumption. In one aspect of the present disclosure, a first composition for reducing the deleterious effects associated with the consumption of alcoholic beverages by an individual is provided. Specifically, and according to some embodiments, the first composition can comprise one or more of the following:
green tea extract;
fructose (e.g., in the form of honey);
vitamin B1;
vitamin B6;
vitamin B12;
pantothenic acid;
vitamin E;
L-cysteine;
zinc;
magnesium; and
milk thistle extract.

In some embodiments, the first composition can further comprise sodium benzoate and/or potassium sorbate.

In another aspect of the present disclosure, a second composition for reducing the deleterious effects associated with the consumption of alcohol beverages by an individual is provided. In some exemplar embodiments, the second composition can comprise one or more of the following:
ginseng;
vitamin C;
glutathione;
vitamin B1 (e.g., in the form of thiamine);
vitamin B2 (e.g., in the form of riboflavin)
vitamin B6;
niacin;
folate;
zinc;
turmeric extract;
ginger root;
magnesium;
caffeine;
fructose; and
pantothenic acid.

In some embodiments, the second composition can further comprise sodium benzoate and/or potassium sorbate.

Disclosed herein are embodiments describing the first and second composition comprising ranges, and specific and preferred proportions of each ingredient within each compound.

In some embodiments, the compositions disclosed herein can be administered orally. For example, some composition embodiments disclosed herein are in aqueous form, for example, as a blended liquid composition. According to one aspect of the embodiments, only one serving or dose is required of each composition to achieve its respective desired effects. In some embodiments, a dose of each composition can be provided as a liquid in a vial that can be consumed as a two-ounce pre-packaged form or shot.

According to another aspect of the embodiments, it is preferred that the first composition is consumed by a drinker prior to, or within, a predetermined time period of alcohol consumption. In some exemplar embodiments, the first composition is consumed by a drinker prior to, or within, six hours of alcohol consumption. According to yet another aspect of the embodiments, it is preferred that the second composition be taken upon ceasing alcohol consumption. Preferably, the second composition should be consumed by a drinker upon onset of symptoms or negative effects associated with alcohol consumption, or within a predetermined time period after waking the day subsequent to alcohol consumption (e.g., within one hour after waking the day subsequent to alcohol consumption.

The first composition and the second composition can be taken individually or in combination with one another. When taken in combination, it is preferred that the first composition is ingested prior to the second composition. Moreover, according to some embodiments, each composition should be taken according to the time parameters described herein. Those of skill in the art will recognize other time intervals in which the first and second composition can be taken to achieve their desired effects.

The ingredients of the first and second compositions are tailored so as to target specific needs during all phases in the metabolic pathway. In this regard, the first and second composition produce optimal results for mitigating and recovering from the effects associated with alcohol consumption. The first and second composition are further tailored so as to work synergistically together at different points in the metabolic pathway so as to more effectively mitigate hangover-related symptoms. Further, the ingredients of the compositions described herein provide hangover remedy and preventative compounds that are nutrient-dense, flavorful, easily administrable, safe to consume, and provide a source of fuel replenishment.

Other compositions, methods, features, and advantages of the subject matter described herein will be or will become apparent to one with skill in the art upon examination of the following detailed description. It is intended that all such additional compositions, methods, features and advantages be included within this description, be within the scope of the subject matter described herein, and be protected by the accompanying claims. In no way should the features of the example embodiments be construed as limiting the appended claims, absent express recitation of those features in the claims.

### DETAILED DESCRIPTION

Provided herein are example embodiments of compositions and methods for alleviating, preventing, and/or mitigating the deleterious effects associated with alcohol consumption. Reference is made to these examples in a non-limiting sense, as it should be noted that they are provided to illustrate more broadly applicable aspects of the composition and/or methods. Various changes may be made to these embodiments and equivalents may be substituted without departing from the true spirit and scope of the various embodiments. In addition, many modifications may be made to adapt a particular situation, material, composition of matter, process, process act(s) or step(s) to the objective(s), spirit or scope of the present disclosure. All such modifications are intended to be within the scope of the claims that can be made herein.

Where a range of values is provided, it is understood that every intervening value, between the upper and lower limit of that range and any other stated or intervening value in the stated range is encompassed within the embodiments described herein. Also, it is contemplated that any optional feature of the inventive variations described may be set forth and claimed independently, or in combination with any one or more of the features described herein. Moreover, no limitations from the specification are intended to be read into any claims, unless those limitations are expressly included in the claims.

As used herein and in the appended claims, the singular forms “a”, “an”, and “the” include plural referents unless the context clearly dictates otherwise. In other words, use of the articles allow for “at least one” of the subject items in the description above as well as the claims below. The claims may exclude any optional element. As such, this statement is intended to serve as antecedent basis for use of such exclusive terminology as “solely,” “only” and the like in connection with the recitation of claim elements, or use of a “negative” limitation.

The subject matter described herein is done so with sufficient detail and clarity to permit the inclusion of claims, at any time, in means-plus-function format pursuant to 35 U.S.C. Section 112, Part (f). However, a claim is to be interpreted as invoking this means-plus-function format only if the phrase “means for” is explicitly recited in that claim.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, acts, steps, or elements of the embodiments may be recited in or added to the claims, as well as negative limitations that define the inventive scope of the claims by features, functions, acts, steps, or elements that are not within that scope.

Aspects of the invention are set out in the independent claims and preferred features are set out in the dependent claims. The preferred features of the dependent claims may be provided in combination in a single embodiment and preferred features of one aspect may be provided in conjunction with other aspects.

A First Embodiment Composition for Mitigating Symptoms Resulting from Alcohol Consumption and Methods Related Thereto

A first embodiment composition for preventing, mitigating, and/or relieving symptoms associated with consumption of alcohol (e.g., ethanol, ethyl alcohol, including but not limited to any beverage, spirit or blend containing ethanol or ethyl alcohol), otherwise known as a “hangover” or “alcohol hangover,” will now be described. The first composition is typically, but not necessarily, ingested or administered as a blended liquid composition. In some embodiments, the blended composition can refer to an intermingled combination of components such as a suspension, emulsion, solution, or mixture. Further, the first composition can take many forms, such as, a dose as a liquid in a vial that can be consumed in a pre-packaged form (e.g., a two-ounce pre-packaged form), a bottle wherein the dose is measured out with a shot glass or pump and then consumed, or a dose pre-added to a liquid beverage, such as syrups, slushies, smoothies, energy drinks, juices, or the like. Those of skill in the art will recognize that the first composition can be ingested and administered through other forms, for example, by capsule, tablet, gel, or powder form, or in a water or other aqueous-mixed solution.

The first composition described herein comprises a various combination of components. Specifically, the blend of the first composition comprises:
green tea extract;
fructose (e.g., honey);
vitamin B1 (e.g., thiamine);
vitamin B6;
vitamin B12;
pantothenic acid;
vitamin E;
L-cysteine;
zinc;
magnesium; and
milk thistle extract.

According to some embodiments of the first composition can comprise the above-mentioned ingredients in the following amounts per serving:
200-600 milligrams (mg) green tea extract per serving;
2-8 grams (g) fructose (e.g., honey) per serving;
2-8 mg vitamin B1 per serving;
2-6 mg vitamin B6 per serving;
500-2000 microgram (mcg) vitamin B12 per serving;
5-200 mg pantothenic acid per serving;
150-600 mg vitamin E per serving;
250-1000 mg L-cysteine per serving;
10-40 mg zinc per serving;
50-200 mg magnesium per serving; and
125-500 mg milk thistle extract per serving.

In some exemplar embodiments, the first composition comprises ingredients in the following proportions per serving, wherein the specific formulation set forth below produces optimal therapeutic results within the permissible deviation indicated above:
400 milligrams (mg) green tea extract;
4 grams (g) fructose (e.g., honey);
4 mg vitamin B1;
3 mg vitamin B6;
1000 microgram (mcg) vitamin B12;
100 mg pantothenic acid;
300 mg vitamin E;
500 mg L-cysteine;
20 mg zinc;
100 mg magnesium; and
250 milk thistle extract.

In other exemplar embodiments, the first composition comprises ingredients in the following proportions per serving, wherein the specific formulation set forth below produces optimal therapeutic results within the permissible deviation indicated above:
400 milligrams (mg) green tea extract;
4 grams (g) fructose (e.g., honey);
2.5 mg vitamin B1 (e.g., thiamine);
3 mg vitamin B6;
1000 microgram (mcg) vitamin B12;
10 mg pantothenic acid;
300 mg vitamin E;
500 mg L-cysteine;
26.6 mg zinc;
100 mg magnesium; and
250 mg milk thistle extract.

Those of skill in the art will recognize that certain individual ingredients can vary in amount, one at a time, or over a wider range and still produce an efficacious composition. However, it is preferred that the ratio of ingredients are consistent with the proportions set forth above.

According to one aspect of the embodiments, the first composition can further comprise one or more preservatives, and/or additives (e.g., natural or artificial flavoring additives). For example, in some exemplar embodiments, the first composition further comprises sodium benzoate and/or potassium sorbate. Potassium sorbate, or C₆H₇KO₂, can refer to a potassium salt of sorbic acid. Potassium sorbate is a chemical additive that is often used as a food or beverage preservative. Sodium benzoate, or C₇H₅NaO₂, can refer to a sodium salt of benzoic acid. Sodium benzoate is often used as a preservative in processed beverages and foods. In some exemplar embodiments, the first composition further comprises 300 parts per million (ppm) of sodium benzoate and 200 ppm of potassium sorbate. Those of skill in the art will recognize that other preservatives and/or natural or artificial flavoring additives can be utilized with the embodiments described herein. For example, those of skill in the art will recognize that additives such as malic acid, calcium disodium EDTA, sucralose, deionized water can be utilized with the embodiments described herein.

Only one serving size or dose of the first composition is required to obtain the desired results. However, one or more serving sizes of the first composition can be ingested by a drinker. The first composition can be served in varying serving sizes. In some embodiments, the serving size of the first composition is about one to two fluid ounces. For example, the first composition can be served as a single two-ounce dose, or as a shot. In other embodiments, the first composition is mixed with an aqueous solution so as to be ingested as a beverage, such as a drink (e.g., a drink between six to sixteen fluid ounces). In this regard, the first composition can be blended with an aqueous solution, such as water or some other hydrating liquid, thereby further mitigating the dehydrating effects associated with alcohol consumption.

In some exemplar embodiments, the first composition should be taken within a predetermined time period (e.g., six hours) of alcohol consumption. Preferably, the first composition should be taken before alcohol consumption. More preferably, a serving of the first composition should be taken at least within a predetermined time window prior to alcohol consumption (e.g., two hours prior to alcohol consumption). In this manner, the first composition can help buffer bodily organs, such as the stomach, kidney, and liver, against the deleterious effects of alcohol consumption and damage caused therefrom. Specifically, by ingesting the first composition in a timely manner, e.g., within six hours of alcohol consumption, it allows the body to more effectively excrete toxins therefrom.

Some of the benefits of the first composition's ingredients will now be described. Green tea extract is an antioxidant and comprises ingredients, such as catechins, theanine, and caffeine. Green tea extract can help alleviate hangover symptoms and prevent serious damage alcohol consumption can cause to organs, such as the liver and stomach, before it occurs.

Honey is comprised of hexose sugars, such as fructose and glucose, e.g., C₆H₁₂O₆. Hypoglycemia, or low blood glucose, can occur as a result of alcohol causing sugar to be lost in the urine during alcohol metabolism. In this regard, hypoglycemia as a result of alcohol consumption can exacerbate hangover symptoms. By consuming honey, the body is given sugar to metabolize in the liver, which not only helps hangover recovery by allowing the body to recover faster from hypoglycemia, but also by allowing the body to burn alcohol faster and excrete alcohol more efficiently.

Vitamin B1, or thiamine, e.g., C₁₂H₁₇N₄OS+, is a water-soluble vitamin of the B complex. Its phosphate derivatives are involved in many cellular processes. The best-characterized form is thiamine pyrophosphate (TPP), a coenzyme in the catabolism of sugars and amino acids. Thiamine is used in the biosynthesis of the neurotransmitter acetylcholine and gamma-aminobutyric acid (GABA). In yeast. TPP is also required in the first step of alcoholic fermentation. Vitamin B1 is readily available from a number of sources, either as a natural or synthetic product, and is generally in a powdered form or liquid, and can be thiamine as the hydrochloride or mononitrate salt. Vitamin B6, or pyridoxine, e.g., C₈H₁₁NO₃ is readily available from a number of sources, either as a natural or synthetic product, and is generally in a powdered form or liquid. Vitamin B12, e.g., C₆₃H₈₈CoN₁₄O₁₄P, is readily available from a number of sources, either as a natural or synthetic product, and is generally in a powdered form or liquid and can be cyanocobalamin. Pantothenic acid is a part of the vitamin B complex and a component of coenzyme A, is readily available from a number of sources, either as a natural or synthetic product, and is generally a viscous liquid. The B vitamins facilitate the process by which an individual's liver can break down alcohol and eliminate it more efficiently.

Vitamin E, as its acetate, is readily available from a number of sources, either as a natural or synthetic product, and is generally in a liquid (viscous oil) form and can be tocopherol. Alcohol promotes oxidative stress and is a major cause of alcohol toxicity in the liver. Vitamin E, as an antioxidant, functions to mitigate the toxic effects associated with alcohol consumption and can help prevent oxidative stress induced by alcohol consumption.

L-cysteine, or cysteine, is an amino acid compound having the chemical formula C₃H₇NO₂S, and can also be in the form of N-acetyl-L-cysteine (NAC). L-cysteine has antioxidant properties and can counteract oxidative stress and inflammatory response associated with alcohol consumption and hangovers.

Zinc is a mineral, and plays a role in producing proteins and enzymes that help metabolize alcohol. Zinc is essential in the conversion of ethanol into acetaldehyde.

Magnesium is a mineral that can have a direct effect on alcohol metabolism. Magnesium provides antioxidant and anti-inflammatory properties which can help alleviate hangover symptoms. Magnesium (e.g., magnesium compounds or magnesium salts) can reduce blood acetaldehyde and alcohol concentrations.

Milk thistle or milk thistle extract is from the seeds of the genus Silybum Adans, which is known for liver protection.

A Second Embodiment Composition for Mitigating and/or Relieving Symptoms Resulting from Alcohol Consumption and Methods Related Thereto

A second embodiment composition for preventing, mitigating, and/or relieving symptoms associated with alcohol consumption will now be described. The second composition is typically, but not necessarily, ingested as a blended liquid composition. Further, the second composition can take many forms, such as, a dose as a liquid in a vial that can be consumed in a \ pre-packaged form (e.g., a two-ounce pre-packaged form), a bottle wherein the dose is measured out with a shot glass or pump and then consumed, or a dose pre-added to a liquid beverage, such as syrups, slushies, smoothies, energy drinks, juices, or the like. Those of skill in the art will recognize that the second composition can be ingested and administered through other forms, for example, by capsule, tablet, gel, or powder form, or in a water or other aqueous-mixed solution.

The second composition described herein comprises a various combination of components. Specifically, the blend of the second composition comprises:
ginseng;
vitamin C;
glutathione;
vitamin B1 (thiamine);
vitamin B2 (riboflavin)
vitamin B6;
niacin;
folate;
zinc;
turmeric extract;
ginger root;
magnesium;
caffeine;
fructose (e.g., honey); and
panthothenic acid.

In some embodiments of the second composition can comprise the above-mentioned ingredients in the following amount per serving:
20-100 mg ginseng per serving;
50-500 mg vitamin C per serving;
250-1000 mg glutathione per serving;
2-8 mg vitamin B1 (e.g., thiamine) per serving;
1-6 mg vitamin B2 (e.g., riboflavin) per serving;
1-6 mg vitamin B6 per serving;
10-50 mg niacin per serving;
200-750 mcg folate per serving;
10-50 mg zinc per serving;
100-500 mg turmeric extract per serving;
20-100 mg ginger root per serving;
50-250 mg magnesium per serving;
50-250 mg caffeine per serving;
5-35 g fructose (e.g., honey) per serving; and
5-200 mg pantothenic acid per serving.

In some exemplar embodiments, the second composition comprises ingredients in the following proportions per serving, wherein the specific formulation set forth below produces optimal therapeutic results within the permissible deviation indicated above:
50 mg ginseng per serving;
100 mg vitamin C per serving;
500 mg glutathione per serving;
4 mg vitamin B1 per serving;
2 mg vitamin B2 per serving;
3 mg vitamin B6 per serving;
20 mg niacin per serving;
400 mcg folate per serving;
20 mg zinc per serving;
250 mg turmeric extract per serving;
50 mg ginger root per serving;
100 mg magnesium per serving;
150 mg caffeine per serving; and
15 g fructose per serving.

In other exemplar embodiments, the second composition comprises ingredients in the following proportions per serving, wherein the specific formulation set forth below produces optimal therapeutic results within the permissible deviation indicated above:
50 mg ginseng per serving;
120 mg vitamin C per serving;
500 mg glutathione per serving;
2.5 mg vitamin B1 per serving;
3.5 mg vitamin B2 per serving;
3 mg vitamin B6 per serving;
26 mg niacin per serving;
450 mcg folate per serving;
20 mg zinc per serving;
150 mg turmeric extract per serving;
50 mg ginger root per serving;
100 mg magnesium per serving;
150 mg caffeine per serving;
20 mg fructose per serving; and
10 mg pantothenic acid.

Those of skill in the art will recognize that certain individual ingredients can vary in amount, one at a time, over a wider range and still produce efficacious composition. However, it is preferred that the ratio of ingredients are consistent with the proportions set forth above.

In some embodiments, the second composition further comprises one or more preservatives. For example, in some exemplar embodiments, the second composition further comprises sodium benzoate and potassium sorbate. Specifically, the second composition further comprises 300 ppm of sodium benzoate and 200 ppm of potassium sorbate. Those of skill in the art will recognize that other preservatives can be utilized with the embodiments described herein.

Only one serving size or dose of the second composition is required to obtain the desired results. However, one or more serving sizes of the second composition can be ingested by a drinker. The second composition can be served in varying serving sizes. In some embodiments, the serving size of the second composition is about one to two fluid ounces. In other embodiments, the second composition is mixed with an aqueous solution so as to be ingested as a beverage, such as a drink (e.g., a drink between six to sixteen fluid ounces). In this regard, the second composition can be blended with an aqueous solution, such as water or some other hydrating liquid, so as to further mitigate the effects of dehydration caused by alcohol consumption.

According to some aspects of the embodiments, the second composition should be taken subsequent to alcohol consumption. The second composition can be taken shortly after the drinker ceases consuming alcohol. Preferably, the second composition should be consumed by a drinker upon the onset of hangover-related symptoms, or within a predetermined time period after waking the day subsequent to alcohol consumption (e.g., one hour after waking the day subsequent to alcohol consumption). In this regard, the second composition helps replenish the body by replacing components lost due to alcohol consumption. Further, the second composition helps the body recover from the negative effects, or hangover-related symptoms, associated with alcohol consumption by ridding the body of toxins more effectively, hydrating the body, and providing a source of energy to recover energy loss associated with alcohol consumption.

According to yet another aspect of the embodiments, the second composition should be taken in combination with the first composition. Specifically, the first composition should be taken prior to the second composition. More specifically, the first composition should be taken prior to alcohol consumption and the second composition should be taken subsequent to alcohol consumption. Even more specifically, the first composition should be taken within six hours of alcohol consumption, while the second composition should be taken the morning following alcohol consumption or at the onset of hangover-related symptoms. In some embodiments, a single serving of the first composition should be taken within six hours of alcohol consumption, and a single serving of the second composition should be taken subsequent to alcohol consumption to achieve their respective desired effects. Further, in this manner, the first composition and second composition work together synergistically at different points in the metabolic pathway so as to more effectively mitigate the negative effects associated with alcohol consumption.

Some of the benefits associated with the ingredients of the second composition have already been described above, with respect to the first composition. Additional benefits associated with additional ingredients in the second composition will not be described.

Ginseng is from a root of plants in the genus Panax, and includes, but is not limited to, Panax Quinquefolius, Panax Ginseng, and Panx Notoginseng. Ginseng can be characterized by the presence of bioactive compounds, such as ginsenosides and gintonin. Ginseng can help reduce blood ethanol concentrations and increase the activity of acetaldehyde dehydrogenase, thereby mitigating hangover-related symptoms.

Vitamin C is readily available from a number of sources, either as a natural or synthetic product, and is generally in a powdered form or liquid and can be ascorbic acid. Vitamin C can help speed up alcohol metabolism by clearing alcohol from a drinker's bloodstream more effectively. Further, vitamin C provides antioxidant and anti-inflammatory properties which help protect a drinker's liver, while also mitigating oxidative stress and neuroinflammation associated with alcohol consumption.

Glutathione is a tripeptide and a major reducing agent in the mammalian body. Glutathione means an amino acid compound of glutamic acid, cysteine and glycine, C₁₀H₁₇H₆N₃O₆S, which can separate into these amino acids by hydrolysis. Glutathione reduces oxidative stress following alcohol consumption and helps eliminate alcohol metabolism's toxic byproducts, such as blood acetaldehyde, which are correlated with hangover symptoms.

Vitamin B2 is readily available from a number of sources, either as a natural or synthetic product, and is generally in a powdered form or liquid and can be riboflavin. Niacin can refer to a nicotinic acid or other compounds having vitamin B3 activity, such as but not limited to nicotinamide, other amide derivatives of nicotinic acid, and nicotinic acid esters. Folate can refer to a folate anion or any compound having vitamin B9 activity. Vitamin B2, niacin, and folate, are each part of the B complex group, and can help facilitate the process by which an individual's liver can break down alcohol and eliminate it more efficiently.

Turmeric extract, or “turmeric, is from the root of Curcuma longa. Turmeric comprises curcuminoids, such as curcumin, C₂₁H₂₀O₆. Turmeric extract and its active ingredients, such as curcumin, provide anti-inflammatory properties and hepatoprotective effects and, as such, can help alleviate the negative effects associated with alcohol consumption and toxicity.

Ginger root, or ginger, is from the root of the Zingiber officinale plant. Gingerol is the main bioactive compound found in ginger and provides anti-inflammatory and antioxidant effects, which can help reduce the oxidative stress associated with alcohol consumption.

Caffeine is a stimulant and an adenosine receptor antagonist, C₈H₁₀N₄O₂. When consumed after alcohol, it can block the nociceptive behaviors associated therewith. Caffeine consumed in combination with anti-inflammatories can block acetate produced by alcohol, thereby relieving hangover symptoms. Caffeine, as provided in the second composition, further provides energy to compensate for energy loss associated with alcohol consumption.

Fructose refers to a monosaccharide, such as glucose. The chemical formula for fructose is C₆H₁₂O₆. Fructose is essential to alcohol metabolism and helps mitigate negative effects associated with alcohol-induced hypoglycemia.

Those of skill in the art will recognize that the ingredients described herein, with respect to the first composition and the second composition, can refer to equivalents of each ingredient known in the art, including but not limited to the products themselves, derivatives, salts, and compounds thereof. Those of skill in the art will further recognize that all amino acids employed in the first and second compositions can be utilized in their natural or L-form. Those of skill in the art will further readily be able to select appropriate materials and methods to manufacture and use the compounds/compositions disclosed herein.

Various aspects of the present subject matter are set forth herein, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible.

1. A composition for mitigating the effects of alcohol consumption, wherein a serving of the composition comprises:
green tea extract;
fructose;
vitamin B1;
vitamin B6;
vitamin B12;
vitamin E;
L-cysteine;
zinc;
pantothenic acid;
magnesium; and
milk thistle extract.

2. The composition of claim 1, wherein the composition further comprises one or more preservatives per serving.

3-4. (canceled)

5. The composition of claim 1, wherein the composition further comprises 400 mg of green tea extract per serving.

6. (canceled)

7. The composition of claim 1, wherein the vitamin B1 includes thiamine, and wherein the composition further comprises 2.5 mg of thiamine per serving.

8. The composition of claim 1, wherein the composition further comprises 3 mg of vitamin B6 per serving and 1000 meg of vitamin B12 per serving.

9. (canceled)

10. The composition of claim 1, wherein the composition further comprises 300 mg of vitamin E per serving.

11. The composition of claim 1, wherein the composition further comprises 500 mg L-cysteine per serving.

12. The composition of claim 1, wherein the composition further comprises 26.6 mg of zinc per serving.

13. The composition of claim 1, wherein the composition further comprises 10 mg of pantothenic acid per serving.

14. The composition of claim 1, wherein the composition further comprises 100 mg of magnesium per serving.

15. The composition of claim 1, wherein the composition further comprises 250 mg of milk thistle extract per serving.

16-17. (canceled)

18. The composition of claim 1, wherein the composition is a blended liquid solution, and wherein the composition is configured to be administered orally.

19-20. (canceled)

21. A composition for mitigating the effects of alcohol consumption, wherein a serving of the composition comprises:
ginseng;
vitamin C;
glutathione;
vitamin B1;
vitamin B2;
vitamin B6;
niacin;
folate;
zinc;
turmeric extract;
ginger root;
magnesium;
caffeine;
fructose; and
panthothenic acid.

22-24. (canceled)

25. The composition of claim 21, wherein the composition further comprises 50 mg of ginseng per serving.

26. The composition of claim 21, wherein the composition further comprises 500 mg of glutathione per serving.

27. The composition of claim 21, wherein the composition further comprises 2.5 mg of vitamin B1, 3.5 mg of vitamin B2, and 3 mg of vitamin B6.

28-29. (canceled)

30. The composition of claim 21, wherein the composition further comprises 26 mg of niacin.

31. The composition of claim 21, wherein the composition further comprises 450 mcg of folate per serving.

32. The composition of claim 21, wherein the composition further comprises 20 mg of zinc per serving.

33. The composition of claim 21, wherein the composition further comprises 150 mg of turmeric extract per serving.

34. The composition of claim 21, wherein the composition further comprises 50 mg of ginger root per serving.

35. The composition of claim 21, wherein the composition further comprises 100 mg of magnesium per serving.

36. The composition of claim 21, wherein the composition further comprises 150 mg of caffeine per serving.

37. The composition of claim 21, wherein the composition further comprises 20 mg of fructose per serving.

38. The composition of claim 21, wherein the composition further comprises 120 mg of vitamin C per serving.

39. The composition of claim 21, wherein the composition further comprises 10 mg of pantothenic acid.

40-41. (canceled)

42. The composition of claim 21, wherein the composition is a blended liquid solution, and wherein the composition is configured to be administered orally.

43-63. (canceled)
